(19) United States
(12) Patent Application Publication
(10) Pub. No.: US 2022/0364143 A1
(43) Pub. Date: Nov. 17, 2022

(54) ANALYSIS OF PROTEIN KINASES IN LIVE CELLS

(71) Applicants: Texas Tech University System, Lubbock, TX (US); University of North Carolina - Chapel Hill, Chapel Hill, NC (US)

(72) Inventors: Degeng Wang, Lubbock, TX (US); Leaf Huang, Durham, NC (US); Hongjun Liang, Lubbock, TX (US); Fengqian Chen, Baltimore, MD (US); Weimin Gao, Morgantown, WV (US)

(21) Appl. No.: 17/741,518

(22) Filed: May 11, 2022

Related U.S. Application Data
(60) Provisional application No. 63/187,965, filed on May 13, 2021.

(51) Int. Cl. C12Q 1/48; C12N 9/12

(52) U.S. Cl. CPC C12Q 1/485; C12N 9/12; G01N 2500/10

(57) ABSTRACT

The present invention includes method and system for detecting kinase activity in vivo, comprising: providing a cell that comprises one or more mutated kinases, wherein the one or more mutated kinases comprise a mutation that enlarges an ATP binding pocket of the kinase; contacting the cell with an ATP analog-nanoparticle conjugate capable of intracellular delivery of the ATP analog-nanoparticle conjugate, wherein the ATP analog comprises a detectable label; culturing the cells under conditions in which the ATP analog-nanoparticle conjugate contacts the one or more mutated kinases in cellulo, wherein the detectable label is transferred from the ATP analog-nanoparticle conjugate to a substrate of the one or more mutated kinases, wherein the one or more kinases react to transfer the detectable label to the substrate.

### CROSS-REFERENCE TO RELATED APPLICATIONS

This application claims priority to U.S. Provisional Application Ser. No. 63/187,965, filed May 13, 2021, the entire contents of which are incorporated herein by reference.

### STATEMENT OF FEDERALLY FUNDED RESEARCH

This invention was made with government support under 1R15GM122006-01A1 and U54 CA198999 awarded by the National Institutes of Health, NIGMS and NCI, respectively. The government has certain rights in the invention.

### TECHNICAL FIELD OF THE INVENTION

The present invention relates in general to the field of protein kinases, and more particularly, to a novel method and system for the analysis of protein kinases in live cells.

### INCORPORATION-BY-REFERENCE OF MATERIALS FILED ON COMPACT DISC

None.

### BACKGROUND OF THE INVENTION

Without limiting the scope of the invention, its background is described in connection with in vitro kinase assays.

Protein kinases function by cleaving the γ-phosphate group off ATP and covalently adding the group onto serine, threonine or tyrosine residues of their substrate proteins, up or down-regulating their activities in vital cellular processes such as cell cycles, growth, migration and apoptosis (1,2). Very often, the substrates themselves are protein kinases. Through this phosphorylation process, the protein kinases form many signaling cascades, e.g., the MAP kinase cascades (3). The kinases and their cascades constitute the backbone of the cellular signaling network, which bridges extracellular and intracellular environment and interconnects subcellular compartments/domains. Many diseases exhibit aberrancies in protein kinases, i.e., over-activation of disease promoting kinases and/or over-repression of disease suppressive ones (4,5). Thus, the treatments often target these kinases. For instance, the type II diabetic treatment targets the AMPK protein kinase (6), and cancer treatments aim to correct the aberrancies of oncogenic and/or tumor-suppressive protein kinases (3,5).

However, kinase study continues to be hindered by a major challenge—insufficient in vivo analysis capability. The tremendous efforts over the last two decades have led to fruitful in vivo activity monitoring for some protein kinases (7-10). Such methods generally work via the expression of a fusion protein composed of a sensing domain, a reporter domain and often a linker region. The sensing domain contains the phosphorylation site of the kinase of interest, phosphorylation of which results in readily quantifiable effects on the activity of the reporter domain. Examples of the effect include activation of FRET, and changes of fluorescence (intensity, wavelength or subcellular locations), activities. However, these strategies have significant drawbacks. First, when homologous kinases share the same substrates, as is often the case, these methods are powerless in differentiating them. Second, they monitor the equilibrium of opposite effects of kinase and phosphatase activities on the sensing domain, instead of solely the kinase activity. Third, a protein kinase often has a large number of substrates with varying affinity to it. The activity on the site chosen for the sensing domain might not reflect faithfully the activities on all of the substrates. Fourth, the methods have no power in identifying new substrates. Thus, in vivo analysis of kinases is far from satisfactory yet.

Currently, kinase studies still rely heavily on one in vitro strategy—incubation of ATP with labeled γ-phosphate group, un-phosphorylated peptide substrate and the kinase in the reaction buffer followed by detecting/quantifying substrate tagging by the labeled γ-phosphate group from ATP. The kinase can be generated through recombinant expression followed by purification and, in case one wishes to quantify an endogenous kinase activity, isolation from cellular extracts. However, it is questionable whether the in vitro substrate tagging reliably reflects the in vivo phosphorylation events and their magnitudes.

Thus, a need remains for novel methods and systems that will permit for the detection of kinase activity in vivo.

### SUMMARY OF THE INVENTION

In one embodiment, the present invention includes a method for detecting kinase activity in vivo, comprising: providing a cell that comprises one or more mutated kinases, wherein the one or more mutated kinases comprise a mutation that enlarges an ATP binding pocket of the kinase; contacting the cell with an ATP analog-nanoparticle conjugate capable of intracellular delivery of the ATP analog-nanoparticle conjugate, wherein the ATP analog comprises a detectable label; and culturing the cells under conditions in which the ATP analog-nanoparticle conjugate contacts the one or more mutated kinases in cellulo, wherein the detectable label is transferred from the ATP analog-nanoparticle conjugate to a substrate of the one or more mutated kinases, wherein the one or more kinases react to transfer the detectable label to the substrate. In one aspect, the cell is a normal cell, a cell clone, a cell line, a transformed cell, or a transfected cell. In another aspect, the ATP binding pocket is enlarged by mutating a gate-keeper Methionine residue into a Glycine. In another aspect, the nanoparticle comprises a liposome, a lipid/calcium/phosphate (LCP) nanoparticle, a polymeric nanoparticle, or a large viral particle. In another aspect, the one or more mutated kinases is transfected into the cell. In another aspect, the substrate is transfected into the cell. In another aspect, the ATP analog is selected from N⁶-phenylethyl-ATP, N⁶-methyl-ATP, N⁶-benzyl-ADP, N⁶-benzyl-ATP, N⁶-phenylpropyl-ATP, or N⁶-phenylbutyl-ATP. In another aspect, the kinase is an adenylate kinase, a tyrosine kinase, a phosphoinositol kinase, a serine/threonine kinase, a single domain kinase, a double domain kinase, a receptor kinase, a histidine kinase, a dual-specificity kinase, a thermostable kinase, or a cytoplasmic kinase. In another aspect, the method determines the presence of the substrate in a sample, wherein the substrate is a known substrate for the one or more kinases, or detecting the presence of a previously unknown substrate for the one or more kinases. In another aspect, the one or more mutated kinases are selected from at least one of: AGC kinases (PKA, PKG, PKC, PKN, PDK1, AKT, SGK, RSK, RSKR, RSKL, GRK, NDR, MAST, DMPK, YANK, and PTF subfamilies), calcium/calmodulin-dependent protein kinases, casein kinase 1, CMGC kinases (CDK, MAPK, GSK3 and CLK subfamilies), NIMA-related kinase (NEK) kinases, receptor guanylate cyclases (RGC), sterile (STE) kinases, tyrosine protein kinase-like (TKL), tyrosine protein kinase (Tyr), aarF-domain containing kinases (ADCK) kinases, Alpha-type kinases, Fas-activated serine/threonine kinase (FAST) kinases, Pyruvate dehydrogenase kinase PDK/BCKDK kinases, PI3/PI4-kinases, or right open reading frame kinases (RIO) kinases. In another aspect, the method further comprises: (i) exposing the kinase coupled to a binding agent specific for the analyte in cellulo, so that a complex is formed between the in vivo kinase and the substrate when present in the cell; (ii) separating complexed in vivo kinase from uncomplexed kinase; wherein the complexed in vivo kinase is contacted simultaneously with ATP and a bioluminescent reagent in cellulo, and (iii) detecting light output from the assay mixture, thereby determining the presence of the analyte in the sample is in cellulo or in vitro. In another aspect, the method further comprises contacting the cell with a kinase inhibitor or a compound suspected of kinase inhibition, and measuring the activity of the in vivo kinase with or without the kinase inhibitor or a compound suspected of kinase inhibition to determine the extent of kinase inhibition. In another aspect, the detectable label is selected from a fluorescence, chemiluminescence, fluorescent energy transfer, radioactive, an enzyme substrate, detection of thiophosphorylation, antigen, or tag.

In another embodiment, the present invention includes an assay for detecting kinase activity in vivo, comprising: providing a cell in a well that comprises one or more mutated kinases, wherein the one or more mutated kinases comprise a mutation that enlarges an ATP binding pocket of the kinase; contacting the cell with an ATP analog-nanoparticle conjugate capable of intracellular delivery of the ATP analog-nanoparticle conjugate, wherein the ATP analog comprises a detectable label; culturing the cells under conditions in which the ATP analog-nanoparticle conjugate contacts the one or more mutated kinases in cellulo, wherein the detectable label is transferred from the ATP analog-nanoparticle conjugate to a substrate of the one or more mutated kinases; and detecting the detectable label on the substrate. In one aspect, the cell is a normal cell, a cell clone, a cell line, a transformed cell, or a transfected cell. In another aspect, the ATP binding pocket is enlarged by mutating a gate-keeper Methionine residue into a Glycine. In another aspect, the nanoparticle comprises a liposome, a lipid/calcium/phosphate (LCP) nanoparticle, a polymeric nanoparticle, or a large viral particle. In another aspect, the one or more mutated kinases is transfected into the cell. In another aspect, the substrate is transfected into the cell. In another aspect, the ATP analog is selected from N⁶-phenylethyl-ATP, N⁶-methyl-ATP, N⁶-benzyl-ADP, N⁶-benzyl-ATP, N⁶-phenylpropyl-ATP, or N⁶-phenylbutyl-ATP. In another aspect, the kinase is an adenylate kinase, a tyrosine kinase, a phosphoinositol kinase, a serine/threonine kinase, a single domain kinase, a double domain kinase, a receptor kinase, a histidine kinase, a dual-specificity kinase, a thermostable kinase, or a cytoplasmic kinase. In another aspect, the method determines the presence of the substrate in a sample, wherein the substrate is a known substrate for the one or more kinases, or detecting the presence of a previously unknown substrate for the one or more kinases. In another aspect, the one or more mutated kinases are selected from at least one of: AGC kinases (PKA, PKG, PKC, PKN, PDK1, AKT, SGK, RSK, RSKR, RSKL, GRK, NDR, MAST, DMPK, YANK, and PTF subfamilies), calcium/calmodulin-dependent protein kinases, casein kinase 1, CMGC kinases (CDK, MAPK, GSK3 and CLK subfamilies), NIMA-related kinase (NEK) kinases, receptor guanylate cyclases (RGC), sterile (STE) kinases, tyrosine protein kinase-like (TKL), tyrosine protein kinase (Tyr), aarF-domain containing kinases (ADCK) kinases, Alpha-type kinases, Fas-activated serine/threonine kinase (FAST) kinases, Pyruvate dehydrogenase kinase PDK/BCKDK kinases, PI3/PI4-kinases, or right open reading frame kinases (RIO) kinases. In another aspect, the method further comprises: (i) exposing the kinase coupled to a binding agent specific for the analyte in cellulo, so that a complex is formed between the in vivo kinase and the substrate when present in the cell; (ii) separating complexed in vivo kinase from uncomplexed kinase; wherein the complexed in vivo kinase is contacted simultaneously with ATP and a bioluminescent reagent in cellulo, and (iii) detecting light output from the assay mixture, thereby determining the presence of the analyte in the sample is in cellulo or in vitro. In another aspect, the assay further comprises contacting the cell with a kinase inhibitor or a compound suspected of kinase inhibition, and measuring the activity of the in vivo kinase with or without the kinase inhibitor or a compound suspected of kinase inhibition to determine the extent of kinase inhibition. In another aspect, the detectable label is selected from a fluorescence, chemiluminescence, fluorescent energy transfer, radioactive, an enzyme substrate, detection of thiophosphorylation, antigen, or tag.

In another embodiment, the present invention includes a system for detecting kinase activity in vivo, comprising: a well that comprises cells that comprise one or more mutated kinases, wherein the one or more mutated kinases comprise a mutation that enlarges an ATP binding pocket of the kinase, wherein the cell is contacted with an ATP analog-nanoparticle conjugate capable of intracellular delivery of the ATP analog-nanoparticle conjugate, wherein the ATP analog comprises a detectable label, and after a predetermined period of time, detecting a detectable label on a substrate from the ATP analog-nanoparticle conjugate contacts the one or more mutated kinases in cellulo, and wherein the detectable label was transferred from the ATP analog-nanoparticle conjugate to the substrate of the one or more mutated kinases. In one aspect, the cell is a normal cell, a cell clone, a cell line, a transformed cell, or a transfected cell. In another aspect, the ATP binding pocket is enlarged by mutating a gate-keeper Methionine residue into a Glycine. In another aspect, the nanoparticle comprises a liposome, a lipid/calcium/phosphate (LCP) nanoparticle, a polymeric nanoparticle, or a large viral particle. In another aspect, the one or more mutated kinases is transfected into the cell. In another aspect, the substrate is transfected into the cell. In another aspect, the ATP analog is selected from N⁶-phenylethyl-ATP, N⁶-methyl-ATP, N⁶-benzyl-ADP, N⁶-benzyl-ATP, N⁶-phenylpropyl-ATP, or N⁶-phenylbutyl-ATP. In another aspect, the kinase is an adenylate kinase, a tyrosine kinase, a phosphoinositol kinase, a serine/threonine kinase, a single domain kinase, a double domain kinase, a receptor kinase, a histidine kinase, a dual-specificity kinase, a thermostable kinase, or a cytoplasmic kinase. In another aspect, the method determines the presence of the substrate in a sample, wherein the substrate is a known substrate for the one or more kinases, or detecting the presence of a previously unknown substrate for the one or more kinases. In another aspect, the one or more mutated kinases are selected from at least one of: AGC kinases (PKA, PKG, PKC, PKN, PDK1, AKT, SGK, RSK, RSKR, RSKL, GRK, NDR, MAST, DMPK, YANK, and PTF subfamilies), calcium/calmodulin-dependent protein kinases, casein kinase 1, CMGC kinases (CDK, MAPK, GSK3 and CLK subfamilies), NIMA-related kinase (NEK) kinases, receptor guanylate cyclases (RGC), sterile (STE) kinases, tyrosine protein kinase-like (TKL), tyrosine protein kinase (Tyr), aarF-domain containing kinases (ADCK) kinases, Alpha-type kinases, Fas-activated serine/threonine kinase (FAST) kinases, Pyruvate dehydrogenase kinase PDK/BCKDK kinases, PI3/PI4-kinases, or right open reading frame kinases (RIO) kinases. In another aspect, the method further comprises: (i) exposing the kinase coupled to a binding agent specific for the analyte in cellulo, so that a complex is formed between the in vivo kinase and the substrate when present in the cell; (ii) separating complexed in vivo kinase from uncomplexed kinase; wherein the complexed in vivo kinase is contacted simultaneously with ATP and a bioluminescent reagent in cellulo, and (iii) detecting light output from the assay mixture, thereby determining the presence of the analyte in the sample is in cellulo or in vitro. In one aspect, the system further comprises a kinase inhibitor or a compound suspected of kinase inhibition that is contacted the cell with, and measuring the activity of the in vivo kinase with or without the kinase inhibitor or a compound suspected of kinase inhibition to determine the extent of kinase inhibition. In another aspect, the detectable label is selected from a fluorescence, chemiluminescence, fluorescent energy transfer, radioactive, an enzyme substrate, detection of thiophosphorylation, antigen, or tag. In another aspect, the system comprises an array comprising two or more wells, wherein each of the wells comprises a different mutated kinase, wherein each well is used to detect an activity of a different mutated kinase on the one or more substrate. In another aspect, the system comprises an array comprising two or more wells, wherein each of the wells comprises a different substrate, wherein each well is used to detect an activity of a different substrate by the one or more mutated kinases.

In another embodiment, the present invention includes a kit for detecting kinase activity in vivo, comprising: a well that comprises cells that comprise one or more mutated kinases, wherein the one or more mutated kinases comprise a mutation that enlarges an ATP binding pocket of the kinase; and an ATP analog-nanoparticle conjugate capable of intracellular delivery of the ATP analog-nanoparticle conjugate, wherein the cell is contacted with the ATP analog-nanoparticle conjugate, and wherein the ATP analog-nanoparticle comprises a detectable label, and after a predetermined period of time, detecting a detectable label on a substrate from the ATP analog-nanoparticle conjugate contacts the one or more mutated kinases in cellulo, and wherein the detectable label was transferred from the ATP analog-nanoparticle conjugate to the substrate of the one or more mutated kinases.

### BRIEF DESCRIPTION OF THE DRAWINGS

For a more complete understanding of the features and advantages of the present invention, reference is now made to the detailed description of the invention along with the accompanying figures and in which:

FIGS. 1A and 1B show an LCP nanoparticle and its synthesis. FIG. 1A: Chemical structures of DOTAP, DOPA, DSPE-PEG and cholesterol. FIG. 1B: Schematic illustration of the synthesis procedure of A*TPγS-loaded LCP nanoparticles.

FIGS. 2A to 2D show the characterization of the LCP nanoparticles. FIG. 2A: Histogram of nanoparticle size. FIG. 2B: Average sizes of blank LCP nanoparticles and A*TPγS-loaded LCP nanoparticles. FIG. 2C: TEM images of A*TPγS-loaded LCP nanoparticles. The experiment was repeated 6 times, a representative result is shown. The scale bar is 100 nm. FIG. 2D: Internalization of Alexa-Fluor-647-ATP-loaded LCP nanoparticles in HCT116 cells. The experiment was repeated 4 times. A representative result is shown. The scale bar is 20.0 μm.

FIG. 3 is a graph that shows the high substrate count of AKT1. A log-log plot of the portion of the kinases with K substrates (P(K)) and the substrate count (K) depicts the scale-free distribution of K, i.e., a linear relationship between log 2(P(K)) and log 2(K). The red * symbol and text illustrate the high Akt1 substrate count. The insert table lists the top 10 high-substrate-count kinases, with AKT1 in bolded text.

FIGS. 4A and 4B show the expression of AKT1 proteins from transfected plasmid vectors. FIG. 4A: HA-tag Western blot analysis of control/untransfected HCT116 cells (C) and HCT116 cells transfected with either the wild type (WT) or the mutant AKT1 expression plasmids (Mutant Akt1). The cells were treated with the A*TPγS-loaded LCP nanoparticles and then lysed. The cell lysates were alkylated by PNBM and subjected to Western blot analysis with the anti-HA-tag antibody. The experiment was repeated more than 3 times. A representative result is shown. FIG. 4B: Transfection and western blot analyses workflow.

FIGS. 5A and 5B show in vivo thiophosphate-tagging of kinase substrates and its detection strategy. FIG. 5A: Wild type kinase utilizes normal ATP to phosphorylate its substrates. In contrast, the mutated kinase, with enlarged ATP binding pocket, accepts bulky A*TPγS and was able to transfer the thiophosphate group onto the substrates. FIG. 5B: Workflow for thiophosphate-labelling of kinase substrates.

FIGS. 6A to 6C show an analysis of in vivo AKT1 auto-thiophosphorylation and thiophosphorylation of AKT1 substrate IKKα. FIG. 6A and FIG. 6B: The transfected HCT116 cell lysates were subjected to anti-thiophosphate-ester antibody immunoprecipitation. This was followed by HA-tag (FIG. 6A) or IKKα (FIG. 6B) Western blot analyses of the control/untransfected HCT116 transfected cell lysate (C) as well as the immunoprecipitants of the lysates of HCT116 cells transfected with either wildtype (WT) or mutant AKT1 (Mutant Akt1) expression plasmids. The same cell lysates as in FIG. 4 were used. Please note that the control/untransfected HCT116 cell lysate was directly used without immunoprecipitation. The experiments was repeated 3 times. A representative result is shown. FIG. 6C: Schematic illustration of the experimental workflow of immunoprecipitation of thiophosphated proteins followed by Western blot analyses.

FIG. 7 is a Western blot analysis of thiophosphorylated proteins. The same cell lysates as in FIGS. 4A-B and 6A-B were used. The PNBM-alkylated cell lysates were subjected to Western blot analysis with the anti-thiophosphate-ester antibody. The experiment was repeated 3 times. A representative result is shown.

FIG. 8 is a Blue silver comassie stained SDS-PAGE gel. The wells for the molecular weight markers (MW), the immunoprecipitant of wild type AKT1 expression plasmid transfected cell lysate (Wild Type) and the immunoprecipitant of mutant AKT1 expression plasmid transfected cell lysate (Mutant) are marked. The antibody (IgG) heavy and light chains are also marked. The experiment was repeated 4 times. A representative result is shown.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The human kinome contains >500 protein kinases, and regulates up to 30% of the proteome. Kinase study is currently hindered by a lack of in vivo analysis approaches due to two factors: our inability to distinguish the kinase reaction of interest from those of other kinases in live cells and the cell impermeability of the ATP analogs. The inventors tackled this issue by combining a widely used chemical genetic method with nanoparticle-mediated intracellular delivery of the ATP analog. As a model system, the critical AKT1 protein kinase was used to demonstrate the present invention. Briefly, enlargement of the ATP binding pocket, by mutating the gate-keeper Methionine residue to a Glycine, prompted the mutant AKT1 to preferentially use the bulky ATP analog N⁶-Benzyl-ATP-γ-S(A*TPγS) and, thus, differentiating AKT1-catalyzed and other phosphorylation events. A lipid/calcium/phosphate (LCP) nanoparticle was used for efficient intracellular delivery of A*TPγS, overcoming the cell impermeability issue. The mutant, but not wild-type, AKT1 used the delivered A*TPγS for autophosphorylation and phosphorylating its substrates in live cells. Thus, an in vivo protein kinase analysis method has been developed. The strategy is widely applicable to other protein kinases.

The present inventors recognized that, in order to establish in vivo kinase analyses in intact live cells, it was necessary to meet two requirements. First, it is necessary to be able to distinguish the kinase reaction of interest from those of the other protein kinases, as the human kinome contains more than 500 protein kinases (2). Second, it was necessary to develop a method to generate sufficient intracellular abundance of the tagged ATP analog. The analog by itself is not cell permeable, but is required as a probe to detect and/or monitor the kinase reactions, i.e., transfer of the γ-phosphate group from ATP onto the substrates.

A chemical-genetic strategy has been used to identify kinase-substrates relationships developed by Dr. Kevan Shokat (11-16). The Shokat method uses site-directed mutagenesis to enlarge a kinase's ATP binding pocket. While not affecting kinase activity, the mutation specifically allows bulky synthetic ATP analogs, such as N⁶-Benzyl-ATP (A*TP), to fit into the enlarged ATP binding pocket (11-16).

A*TP can be further modified to create A*TP-γ-S by replacing an O with a S in the γ-phosphate group. The mutant protein kinase preferentially use A*TP-γ-S in its kinase reaction, transferring the thiophosphate tag from A*TP-γ-S onto its substrate proteins. This tag can then be alkylated by a chemical called p-nitrobenzylmesylate (PNBM) to generate a thiophosphate-ester for specific antibody recognition or affinity purification followed by LC-MS analysis (17-19). That is, this method combines ATP-binding pocket enlargement and the bulky ATP analog A*TP-gamma-S to distinguish the kinase reaction of interest from those of the other protein kinases, enabling identification of the substrates of specific kinases. However, due to the cell impermeability of the A*TP-γ-S molecule, this method is limited to in vitro kinase activity with cell lysate in test tubes.

Thus, the present inventors recognized that in order for the thiophosphate-tagging of the substrates to occur in live cells, it was also necessary to accomplish the second requirement, i.e., a reliable method for efficient intracellular A*TP-γ-S delivery. A nanoparticle delivery approach was adapted for this purpose. A lipid/calcium/phosphate (LCP) nanoparticle includes a calcium phosphate (CaP) core, which carries the to-be-delivered chemicals, and a lipid bilayer, which encapsulates the CaP core and enables cellular uptake of the nanoparticle through endocytosis. This nanoparticle has been used for efficient intracellular delivery of Gemcitabine Triphosphate, a nucleotide analog used as a chemotherapy agent (20,21). The A*TP-γ-S molecule is chemically similar to Gemcitabine Triphosphate. Thus, the inventors became interested in using the LCP nanoparticle as a vehicle for intracellular A*TP-γ-S delivery and, in turn, to enable intracellular thiophosphate-tagging of protein kinase substrates via the Shokat chemical genetic approach; that is, achievement of in vivo protein kinase analysis via a combination of the Shokat chemical genetic method and the nanoparticle delivery of the A*TP-γ-S molecule.

To accomplish this goal, the inventors chose the serine/threonine protein kinase AKT1 (also known as PKB) as the initial prototype. Firstly, AKT1 is functionally and therapeutically important (22). It is part of the critical phosphoinositide 3-kinase (PI3K) signaling module (23-27). The non-receptor tyrosine kinase ACK1/TNK2 can also phosphorylate AKT1, resulting in PI3K-independent activation (28). AKT1 phosphorylates and regulates a wide range of metabolic and/or regulatory substrate proteins such as IKKalpha, GSK3beta, and FOXO (29,30). Thus, it participates in multiple vital cellular processes, including cell survival and cell growth. As the Akt signaling process aberrancy occurs in multiple human diseases such as diabetes and cancer, AKT1 is a significant therapeutic target for multiple human diseases (22, 24, 29, 31-36). Secondly, perhaps due to the functional importance, AKT1 is one of the earliest protein kinases studied successfully with the chemical genetic approach. Moreover, in addition to phosphorylating its numerous substrates, AKT1 undergoes auto-phosphorylation (40).

Thus, the inventors enlarged the AKT1 ATP binding pocket by mutating the gate-keeper Methionine residue into a Glycine. The inventors also used the LCP nanoparticle for efficient intracellular delivery of the A*TPγS molecule, overcoming the cell impermeability issue. It is demonstrated herein that the mutant, but not the wild type, AKT1 was able to use the delivered A*TPγS for autophosphorylation as well as phosphorylating other protein substrates in live cells. In a word, an in vivo AKT1 kinase analysis method has been developed, to the best of the inventors' knowledge, for the first time. Further, this novel strategy uses only basic commodity reagents and equipment and is widely applicable to other protein kinases.

Non-limiting examples of kinases that can be modified and tested using the present invention can be found at: kinase.com/kinbase/FastaFiles/human_protein_sequences.fasta, amino acid sequences incorporated herein by reference. A complete list of kinases can be found at kinase.com/human/kinome, relevant nucleic and amino acid sequences taught therein are incorporated herein by reference. Examples of kinases for use with the present invention include adenylate kinase, a tyrosine kinase, a phosphoinositol kinase, a serine/threonine kinase, a single domain kinase, a double domain kinase, a receptor kinase, a histidine kinase, a dual-specificity kinase, a thermostable kinase, or a cytoplasmic kinase. More particular examples of kinases that can be mutated to enlarge the ATP binding pocket can be selected from at least one of: AGC kinases (PKA, PKG, PKC, PKN, PDK1, AKT, SGK, RSK, RSKR, RSKL, GRK, NDR, MAST, DMPK, YANK, and PTF subfamilies), calcium/calmodulin-dependent protein kinases, casein kinase 1, CMGC kinases (CDK, MAPK, GSK3 and CLK subfamilies), NIMA-related kinase (NEK) kinases, receptor guanylate cyclases (RGC), sterile (STE) kinases, tyrosine protein kinase-like (TKL), tyrosine protein kinase (Tyr), aarF-domain containing kinases (ADCK) kinases, Alpha-type kinases, Fas-activated serine/threonine kinase (FAST) kinases, Pyruvate dehydrogenase kinase PDK/BCKDK kinases, PI3/PI4-kinases, or right open reading frame kinases (RIO) kinases.

Materials. Alexa-Fluor-647-ATP was obtained from Thermo Fisher Scientific (Massachusetts, USA), and N⁶—Benzyladenosine-5′-(3-thiotriphosphate) (A*TPγS) was obtained from BIOLOG Life Science Institute (Bremen, Germany). IKK alpha monoclonal antibody was obtained from Thermo Fisher Scientific (Massachusetts, USA), anti-thiophosphate ester antibody was obtained from Abcam (Cambridge, UK), and AccuRuler RGB protein ladder and BP-Fectin mammalian cell transfection reagent were obtained from BioPioneer (California, USA). Dioleoyl phosphatydic acid (DOPA), 1,2-dimyristoyl-3-trimethylammonium-propane chloride salt (DOTAP), and 1,2-di stearoyl-sn-glycero-3-phosphoethanolamine-N-[amino(polyethylene glycol)-2000] ammonium salt (DSPE-PEG₂₀₀₀) were obtained from Avanti Polar Lipids, Inc. (Alabama, USA). DSPE-PEG-anisamide (AEAA) was synthesized as described previously (41). Other chemicals were purchased from Sigma-Aldrich, Inc. (Missouri, USA).

Cell culture. HCT116 human colon cancer cells, originally obtained from Dr. Michael Brattain's lab (42,43), were cultured in the McCoy's 5A medium (Invitrogen, USA) supplemented with 10% fetal bovine serum, 100 U/mL penicillin, and 100 mg/mL streptomycin (Invitrogen, USA). Cells were cultivated at 37° C. and 5% CO₂ in an incubator.

Preparation of A*TPγS-loaded LCP nanoparticles. Briefly, 100 μL of 10 mM specified ATP analog solution (A*TPγS or Alexa-Fluor-647-ATP) in 12.5 mM Na₂HPO₄ solution and 100 μL of 2.5 M CaCl₂) solution were dispersed in cyclohexane/Igepal solution (70/30, v/v), respectively, to form an oil phase. After 15 min of stirring, 100 μL of 25 mM DOPA was added into the oil phase for 15 minutes, then 40 mL of 100% ethanol was added into the oil phase. Later, the mixture solution was centrifuged at 10,000 g for 20 minutes to pellet the LCP particle core out of the supernatant solution. After washing with 50 mL ethanol twice, 100 μL of 10 mg/mL cholesterol, 100 μL of 25 mg/mL DOTAP, 100 μL of 25 mg/ml DSPE-PEG, and 10 μL of 25 mg/mL DSPE-PEG-AEAA were added into the LCP core solution. After evaporating under N₂, the residual lipids were suspended in PBS to produce the layer of LCP nanoparticle. After being sonicated for 10 minutes, the LCP solution was dialyzed in PBS.

Characterization of A*TPγS-loaded LCP nanoparticles. The LCP nanoparticle particle sizes was determined by Malvern DLS (Royston, UK). The concentration of A*TPγS was determined by an HPLC spectrophotometer (Shimadzu Corp., Japan). Transmission electron microscope (TEM) photos of A*TPγS-loaded LCP nanoparticles were observed under JEOL 100CX II TEM (Tokyo, Japan).

Intracellular uptake behaviors. Intracellular uptake behaviors were observed with a confocal microscopy. Briefly, the HCT116 cells were plated into confocal dishes (1×10⁵ cells per well) and incubated for 24 hours. Then, the cells were incubated with Alex-Fluor-647-ATP-loaded LCP nanoparticles for 4 hours with a concentration of 5 μg/mL, and were nucleus-labeled with Hoechst 33342 dye (Thermo Fisher Scientific) for 15 minutes. The images were taken with a Zeiss 880 confocal microscope (Germany).

Plasmid constructs. Wild type AKT1 plasmid was purchased from Addgene (Addgene plasmid pcDNA3-myr-HA-AKT1 1036). Point mutation as in references (37) of the ATP-binding pocket gatekeeper amino acid (M227G) were introduced by GENEWIZ (New Jersey, USA), with a modified Stratgene QuikChange® site-directed mutagenesis method.

Expression plasmid transfection. The HCT116 cells were plated into 100 mm² dishes and transfected at approximately 90% confluence with the specified plasmid (wild type pcDNA3-myr-HA-AKT1 plasmid or the mutant pcDNA3-myr-HA-AKT1 plasmid) using BP-Fectin mammalian cell transfection reagent (BioPioneer, USA) in accordance with the manufacturer's instructions. One untransected dish was used as a negative control. After 72 hours, cells were washed twice with cold PBS. Transfection efficacy was confirmed by western-blot measurement of the expression of the HA-tagged fusion proteins with the HA tag monoclonal antibody (Thermo Fisher Scientific, USA).

Intracellular A*TPγS delivery and thio-phosphorylation assays. Control and transfected HCT116 cells were incubated with A*TPγS-loaded LCP nanoparticles for 4 hours. The cells were washed by cold PBS twice and then lysed with RIPA buffer (which contains protease and phosphatase inhibitors) for 30 minutes at 4° C. The cell lystaes were treated with 2.5 mM PNBM at room temperature for 1 hour. Then, the PNBM treated cell lysates were analyzed with a western blot assay to measure protein thio-phosphorylation with the anti-thiophosphate ester rabbit monoclonal antibodies (Abcam, USA).

Immunoprecipitation assays. The PNBM-treated cell lysates were pre-cleaned by an off-target antibody. Then the cleaned cell lysates were incubated with 10 μL anti-thiophosphate ester rabbit monoclonal antibody (Abcam, USA) at 4° C. for 12 hours. 100 μL protein G beads (Thermo Fisher Scientific, USA) were mixed under rotary agitation at 4° C. for 4 hours, washed twice by wash buffer, and the beads were collected. Then, proteins were captured in the beads and released by elution buffer. The elution buffer was collected and stored at −80° C.

Western blotting assay. First, total protein concentrations of the cell lysates were determined by a BCA protein assay kit (Thermo, USA). Protein extracts were separated by SDS-PAGE, then transferred onto a nitrocellulose membrane (NC membrane). After blocking the NC membrane with 5% non-fat milk for 1 hour at room temperature, it was incubated with the specified first antibody (the HA tag monoclonal antibody, the anti-thiophosphate ester antibody or the IKK alpha monoclonal antibody) at 1:1000 dilution at 4° C. overnight. Membranes were washed five times with TBST solution, incubated with the corresponding secondary antibody (1:3000 dilution) for 1.5 hours at normal room temperature, and washed five times with TBST solution. Protein bands were visualized using an enhanced chemiluminescence system according to the manufacturer's instructions (Thermo Fisher Scientific, USA).

PhosphoSitePlus dataset analysis. In order to perform human kinome-wide analysis, the PhosphoSitePlus dataset was downloaded from the database website (www.phosphosite.org). For this study, all non-human protein kinases and substrates were excluded. Substrate count was then calculated for each kinase with the remaining data.

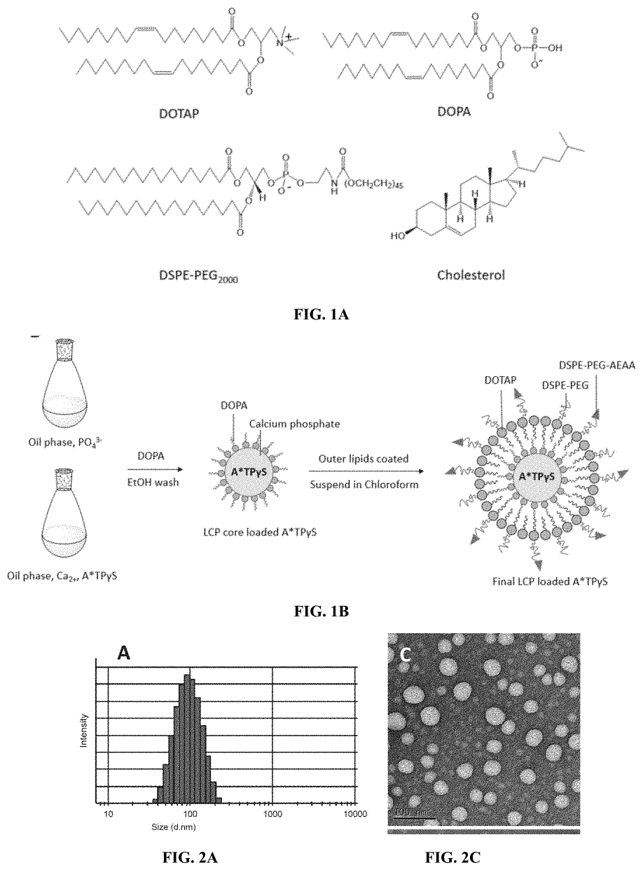

Characterization of A*TPγS-loaded LCP Nanoparticles. As mentioned earlier, the LCP nanoparticle is a formulation that has been used for efficient intracellular delivery of the nucleotide analog Gemcitabine Triphosphate as a chemotherapy agent. Herein, the inventors tested it for intracellular A*TPγS delivery to overcome the cell impermeability issue. FIG. 1A: Chemical structures of DOTAP, DOPA, DSPE-PEG and cholesterol. FIG. 1B shows a schematic illustration of the preparation procedure of A*TPγS-loaded LCP nanoparticles. A*TPγS is first loaded into the CaP core of the LCP nanoparticles, which is then coated with a DOPA layer. DOPA functions to prevent the aggregation of the CaP core because of the strong interaction between the CaP core and the phosphate head of DOPA. The cationic 1,2-dioleoyl-3-trimethylammonium-propane (DOTAP) lipid is then added as an outer layer onto the DOPA layer (21). Additionally, an outside layer of neutral dioleoylphosphatidylcholine (DOPC) with DSPE-PEG with a tethered targeting ligand anisamide (AA) is added. These layers keep the nanoparticles stable in the hydrophilic tissue culture medium and also enhance cellular uptake of the nanoparticles. Upon nanoparticle cellular internalization, the CaP core is rapidly dissolved in the endosome due to its low pH, leading to increased endosome osmotic pressure. Consequently, the endosome swells and ruptures, releasing the loaded A*TPγS into the cytoplasm. This quick process also avoids lysosomal degradation after endocytosis.

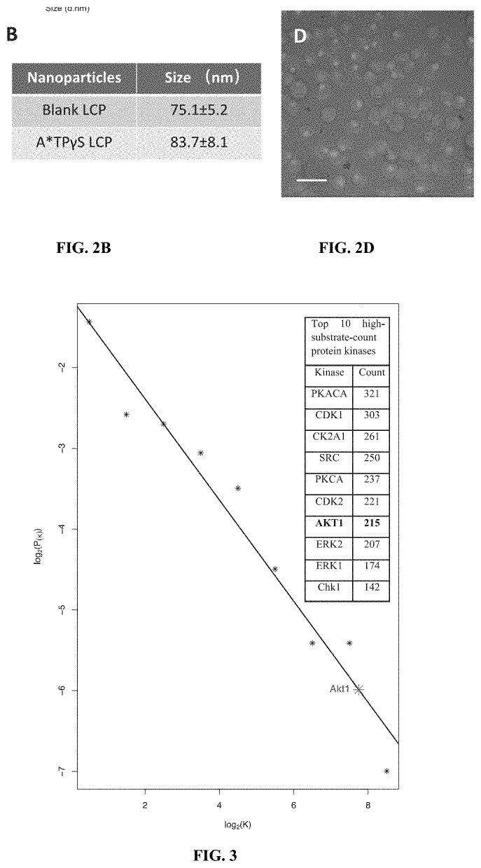

FIGS. 2A to 2D summarize the results of our characterization of synthesized nanoparticles. The loaded LCP nanoparticle sizes are exhibited in FIG. 2A in a histogram. The sizes are also summarized in FIG. 2B along with those of unloaded nanoparticles. On average, A*TPγS-loaded LCP nanoparticles were 83.7±8.1 nm and, as expected, about 11% larger than unloaded nanoparticles, which has a size of 75.1±5.2 nm. A transmission electron microscope (TEM) image is also shown in FIG. 2C, illustrating the monodispersed nanoparticles with spherical shapes. The final concentration of A*TPγS in 1 mL of produced nanoparticles was 0.5 mM. Thus, about 50% of the starting A*TPγS (100 μL of 10 mM solution) was loaded into the nanoparticles.

The inventors also monitored intracellular uptake of the LCP nanoparticle by using the fluorescent ATP analog Alexa-Fluor-647-ATP. As described in Materials and Methods, this ATP analog was loaded into the LCP nanoparticles. The loaded nanoparticle was added into the tissue culture medium and incubated with the HCT116 cells for 4 hours for intracellular uptake. The cells were then nucleus-stained and observed under a confocal microscope. The result is shown in FIG. 2D. Strong Alexa-Fluor-647 red fluorescence activities were spotted inside the HCT116 cells, demonstrating the cellular internalization of loaded LCP nanoparticles. Thus, the inventors demonstrated herein that the LCP nanoparticles could be used as a vehicle for intracellular A*TPγS delivery.

Generation and expression of mutant AKT1 kinase. The inventors generated other components of the Shokat chemical genetic method, in order to combine it with nanoparticle-mediated intracellular A*TPγS delivery to enable in vivo protein kinase substrate detection.

First, as briefly mentioned earlier, the AKT1 protein kinase was chosen as our initial prototype to develop an in vivo method for identifying protein kinase-substrates relationship due to feasibility and functional importance. The AKT1 mutant with enlarged ATP binding pocket has been shown to bind to the bulky ATP analogue A*TP. The mutant retained the protein kinase activity and exhibited the same ectopic expression level as the wild type AKT1 kinase (37,38). It was also reported by Dr. Shokat's group that ATPγS (note: not A*TPγS) can be utilized by wild type AKT1 to tag its substrate GSK3β in vitro (44). In a word, these evidences support AKT1 as the most feasible choice of initial prototype. Moreover, FIG. 3 illustrated quantitatively the functional importance of AKT1 in terms of the number of experimentally determined AKT1 subtrates. The PhosphoSitePlus database (www.phosphosite.org) dataset was used and the number of unique experimentally verified substrates for each human protein kinases were counted, revealing that human AKT1 has 215 unique substrates (45). As many other cell biology parameters (46-49), the substrate count (K) of the human kinome follows the so-called scale-free distribution, i.e., P₍ₖ₎∝K^{-α} or log₂(P₍ₖ₎)∝−α*log₂(K), with P_{(K)} as the portion of protein kinases with K unique substrates and α as a positive constant. This is illustrated in the linear relationship in a log-log plot (log₂(P₍ₖ₎ vs. log₂(K)) in FIG. 3. A small number of protein kinases have extra-ordinarily high substrate counts, while the majority of the kinases have only a small number of substrates. As shown by the red symbol in FIG. 3, AKT1 is one of the high-substrate-count kinases; it was ranked, as shown in the insert table in FIG. 3, at the 7^{th} among all human protein kinases. Therefore, AKT1 is not only the most feasible, but also a functionally important, choice for our study.

The pCDNA3-Myr-HA-AKT1 expression plasmid from AddGene was used. The vector expresses the AKT1 protein fused to the HA and the Myr tags. The HA-tag enables detection of the expressed AKT1 protein with the HA-tag antibody, facilitating transfection analysis. The Myr tag renders the expressed protein myristoylated, membrane anchored and, thus, contitutively active. The inventors applied site-directed mutagenesis to mutate the gatekeeper Methionine codon into a Glycine codon in AKT1 ATP binding pocket (37). The two vectors made it possible for us to test whether the bulky A*TPγS molecule is too large for AKT1 and other wild type protein kinases, but fits into the enlarged ATP binding pocket of mutant AKT1 and leads to specific thiophosphorylation of AKT1 substrates.

Second, to test thio-phosphate-tagging of substrate proteins in live cells, a host cell line was needed. The human HCT116 cell line was chosen. This cell line has an activating point mutation in the PI3KCA gene (50,51), resulting in constitutive PI3K kinase activity and thus elevated activation of the expressed AKT1 fusion proteins, further facilitating in vivo detection of thio-phosphate-tagging of AKT1 substrates.

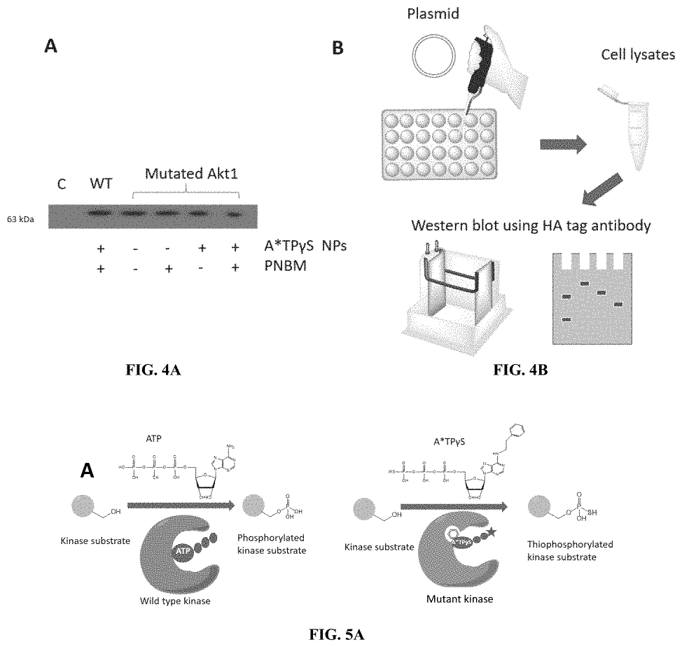

Having established these components, the inventors next tested the expression of both wildtype and mutant AKT1 proteins upon transfection of the respective expression plasmids into the HCT116 cells and subsequent treatments. 72 hours after the tranfection, the cells were treated with the A*TPγS-loaded nanoparticle for 4 hours. The cells were then lysed. The cell lysates were treated with the PNBM alkylation to convert the thiophosphate tag into thiophosphate-ester tag, and then aliquoted. The inventors then performed Western blot analysis with one aliquote set of the cell lysates and the HA-tag antibody to measure the ectopic expression of wildtype and mutant AKT1 proteins. As shown in FIG. 4A, both wild type and mutant AKT1 expression vectors drove strong expression of the HA-tagged proteins in the HCT116 cells upon transfection, while the control/untransfected HCT116 cells exhibited no detectable HA-tagged AKT1 expression. The A*TPγS-loaded nanoparticle and the PNBM treatments did not affect the expression significantly. The other aliquots of the same cell lysates were used, as described below, in subsequent downstream analyses.

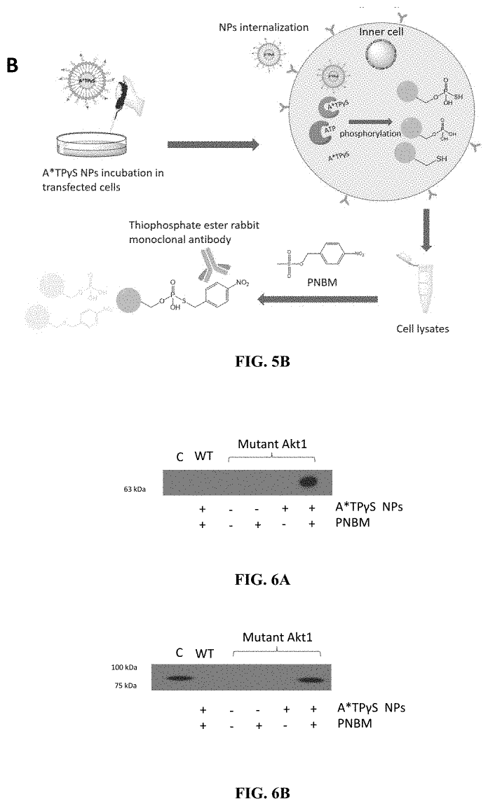

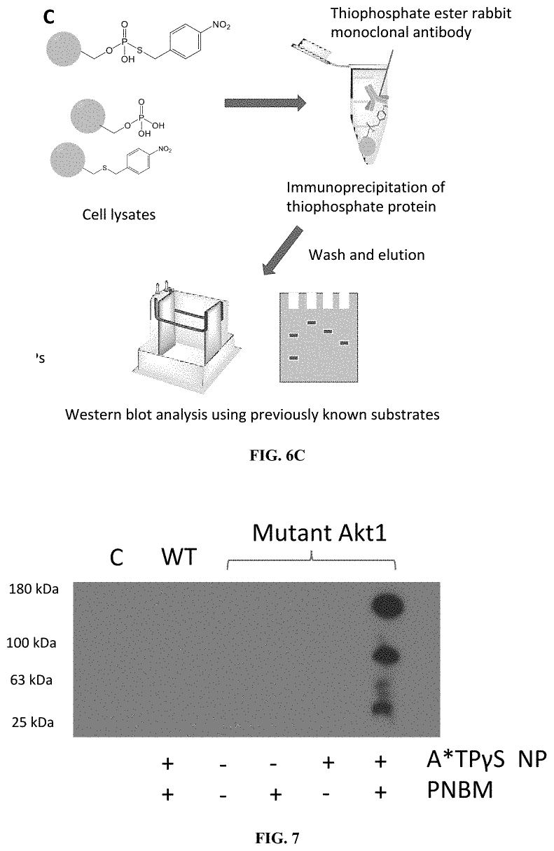

Specific in vivo utilization of A*TPγS by mutant AKT1. Next, the inventors determined whether the mutant AKT1 protein can use the nanoparticle-delivered A*TPγS in its phosphorylation reaction, i.e., transferring the γ-thiophosphate group onto AKT1 substrates (FIGS. 5A-B). For this purpose, the inventors tested potential AKT1 self-tagging through its autophosphorylation activity as well as tagging of another well-known AKT1 substrate, the IKKalpha protein (30). Since alkylation by PNBM should have converted the thiophosphate tag into the thiophosphate ester tag (52), the anti-thiophosphate ester monoclonal antibody should be able to recognize the tagged proteins. Thus, another aliquot set of the treated cell lysates was used for the tests. The lysates of the transfected cells were subjected to immunoprecipitation with the thiophosphate ester tag antibody. The immunoprecipitants, along with the un-immunoprecipitated lysate of control/untransfected cells, are then examined by Western blot analyses with HA-tag or IKKalpha antibodies; the un-immunoprecipitated control/untransfected cell lysate served as negative and positive controls for the HA-tag and the IKKalpha antibodies, respectively. The results are shown in FIGS. 6A to 6C. The HA-tag antibody was used in this analysis to detect self-tagging of the Myr-HA-AKT1 fusion proteins due to AKT1 auto-phosphorylation. As shown in FIG. 6A, the fusion protein was detected in the immunoprecipitant of the cells transfected with the mutant AKT1 expression vector. Both nanoparticle treatment of the cells and PNBM alkylation of the cell lysate were required; the bands disappeared when either treatment was not performed. However, the fusion protein was not detected in the immunoprecipitants of the cells transfected with wild type AKT1 expression vector, even though the cells were treated with A*TPγS-loaded LCP nanoparticle and the cell lysate was also treated with PNBM. Not surprisingly, the control/untransfected cell lysate gave no band due to a lack of expression of the HA-tagged fusion protein. The same was observed for the IKKalpha protein (FIG. 6B). IKKalpha was detected with the IKKalpha antibody in the immunoprecipitants of the cells transfected with the mutant AKT1 expression vector, but not in that of the cells transfected with wild type AKT1 expression vector. As expected, the antibody was able to detect endogenous IKKalpha protein in the un-immunoprecipitated control/untransfected HCT116 cell lysate. Thus, the mutant AKT1 protein was able to use, exclusively, the nanoparticle delivered intracellular A*TPγS for in vivo thiophosphate-tagging of its substrates in live HCT116 cells.

AKT1 is known to phosphorylate a large number of substrates. As discussed earlier, a search of the PhosphoSitePlus database (www.phosphosite.org) revealed that human AKT1 has 215 unique human proteins as its experimentally verified substrates and was the 7^{th} human kinase with most substrates (45). Thus, the inventors determined whether the mutant AKT1 protein would use the delivered A*TPγS to tag many other proteins in the HCT116 cells. First, the inventors used Western blot with the thiophosphate-ester antibody to detect the presence of tagged proteins in another aliquot set of the cell lysates, which had been used in FIGS. 4A-B and 6A-C. As shown in FIG. 7, only the cells that were transfected with the mutant AKT1 expression vector and treated with A*TPγS-loaded nanoparticles and whose cell lysates were PNBM-alkylated exhibited bands in the Western blot analysis (FIG. 7). Multiple prominent bands were detected, ranging from 25 to 180 kDa. This is not surprising, given that AKT1 is known to phosphorylate a large number of substrates. As expected, wild type AKT1 was not able to use the bulky A*TPγS, although the cell lysate was also treated with PNBM. Once again, for cells transfected with the mutated AKT1 expression vector, both nanoparticle treatment of the cells and PNBM alkylation of the cell lysate were required; the bands disappeared when either treatment was not performed.

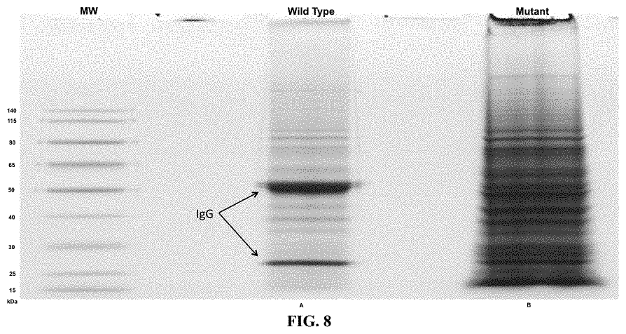

Second, the inventors performed immunoprecipitation of the PNBM alkylated lysates of cells transfected with expression vectors and treated with the nanoparticle. The immunoprecipitants were subjected to SDS-PAGE electrophoresis. The gel was then silver blue comassie stained, in order to compare the protein abundance in the two immunoprecipitants. As expected, the thiophosphate ester antibody was able to immnoprecipitate at high quantities a large number of proteins from the lysate of mutant-AKT1-expressing cells, but not from that of wild-type-AKT1-expressing cells (FIG. 8). Taken together, the results of the two experiments suggest that LCP-nanoparticle delivered intracellular A*TPγS can be used exclusively by the mutant AKT1 protein to thiophosphate-tag its substrates in live cells. That is, by combining nanoparticle-mediated intracellular A*TPγS delivery and the chemical genetic method, an in vivo approach for kinase-substrate relationship determination has been established.

Until now the study of protein kinases has relied heavily on in vitro experimental protocols due to the cell impermeability of key analysis probes—the ATP analogs—and the inability to distinguish kinase reactions from one another in live cells. The present invention has overcome these problems. Nanoparticle-mediated intracellular delivery of A*TPγS overcomed the cell impermeability issue. Using the AKT1 protein kinase as the initial prototype, delivered A*TPγS enabled in vivo substrate thiophosphate tagging by mutant AKT1 with enlarged ATP binding pocket, which was created according to the Shokat chemical genetic method. Consequently, the inventors were able to detect kinase-substrate relationship in live cells and in a kinase-specific manner.

The results pave the way to unbiased mass-spectrometry (MS) based proteomic study of kinase-substrate relationship. The AKT1 protein kinase, as discussed earlier, has a large number of substrates. As shown in FIG. 8, the thiophosphate ester antibody was able to immunoprecipitate a large number of tagged proteins from the lysate of HCT116 cells expressing the mutant AKT1 protein. Thus, the present invention allows for the first time comparative MS analysis of the immunoprecipitant with that of wildtype-AKT1-expressing cells will lead to unbiased identification of both known and new AKT1 substrates.

The method and system taught herein is applicable to the other protein kinases due to the conservation of the ATP binding pocket across the kinome. The chemical genetic method is widely applicable. It has been applied to a large number of human protein kinases; just to name a few, EGF receptor, SRC, p38/MAPK14 and ERK1/2 (39). Sometimes, a different derivative of the ATP molecular was used. Consequently, the nanoparticle-mediated intracellular delivery of the bulky ATP analogs should be applicable in enabling in vivo analysis of these protein kinases as well.

The chemical genetic approach has been applied to study other ATP binding proteins (53). The DDX3 gene codes for a DEAD-box protein and is frequently mutated in human cancers. The DDX3 protein belongs to a large family of ATP-dependent RNA chaperones. The ATP binding pockets of the DEAD-box proteins in this family are very similar to one another, making it challenging to develop DDX3-specific inhibitors. It was shown that mutating the hydrophobic amino acid residues, which interact with ATP N6 position in the binding pocket, enabled the mutant DDX3 protein to bind to bulky ATP analogs (53). The strategy of using nanoparticle-mediated intracellular delivery of the present invention ensures efficient delivery of the bulky ATP analog that can now be used with mutant-DDX3-specific inhibitors.

Moreover, the present invention can use other derivatives of the A*TP (N6-Benzyl-ATP) molecule for in vivo probing of other aspects of protein kinase function. A*TPγS derives from A*TP by substituting the γ-phosphate group with a thiophosphate group. The purpose of the thiophosphate group is to act as a handle for kinase substrate identification.

The present invention can also be used for important aspects of kinase study, e.g., ATP binding and enzymatic activities. The ATP binding pocket is usually shielded from ATP binding in an inactive protein kinase, but becomes accessible upon activation of the kinase. For instance, in monomeric inactive EGF receptor (EGFR), the activation loop is in close proximity of the pocket and blocks ATP binding. Upon ligand binding and dimerization, the C-lobe of one EGFR molecule disrupt this autoinhibitory conformation of the other EGFR molecule, enabling ATP binding and kinase activation (54). However, current ATP binding and kinase activity analyses rely on the usage of ATP analogs, and cell impermeability of these analogs renders in vivo analyses impossible. Using the approach taught herein for intracellular delivery of relevant A*TP derivatives overcome this cell impermeability obstacle.

Nanoparticles have been widely used for therapeutic agent delivery in biomedical researches. For instance, the LCP nanoparticle has been used to deliver gene therapy agents such as siRNA and expression vectors (55,56). It has also been used to deliver many therapeutical chemicals such as gemcitabine triphosphate (20,21), doxorubicin and paclitaxel (57). Due the similarity between A*TPγS and gemcitabine triphosphate, the inventors chose to use this nanoparticle for intracellular A*TPγS delivery in this study. To the best of the inventors' knowledge, the present invention represents a new application of nanoparticles—overcoming cell impeameability of key analysis probes to enable in vivo execution of previously in vitro experimental procedures. The principle is applicable to a wide variety of biochemical, molecular and cellular research techniques, perhaps adopting difference types of nanoparticles for different purposes. Using the present invention previously impossible research opportunities are now available.

In summary, in this study, the inventors developed an in vivo protein kinase analysis by successfully combining nanoparticle-mediated probe (A*TPγS) delivery and a chemical genetic method. The former overcome the A*TPγS cell impermeability issue, and the latter enabled differentiation of the kinase reaction of interest from those of other protein kinases. Using the AKT1 model system, the inventors achieved detection of kinase-substrate relationships in live cells. This approach can be applied to other protein kinases and other aspects of protein kinase studies. Nanoparticle-mediated delivery of key analysis probes can be used to overcome cell impermeability and is generally applicable to many other biochemical, molecular and cellular biomedical research areas.

Identification of the in vivo substrates of specific protein kinases in intact cells offers a great understanding of intracellular signaling pathways and benefits therapeutic targets for human diseases (2). Herein, the inventors have developed a strategy to exclusively tag the direct substrates of a protein kinase of interest in intact cells, combining the chemical genetics method and the nanoparticle delivery system to uniquely transfer γ-thiophosphorylation tagging to AKT1 substates in intact cells. Specifically, the inventors confirmed using an AKT1 substrate using immunoprecipitation and Western blot assays. Finally, this in vivo substrate identification strategy can identify both known and novel substrates, offers a novel route to identify substrates of various kinases, and can be applied to the signaling cascade study.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Dancey, J. E., Bedard, P. L., Onetto, N. and Hudson, T. J. (2012) The genetic basis for cancer treatment decisions. Cell, 148, 409-420.
2. Manning, G., Whyte, D. B., Martinez, R., Hunter, T. and Sudarsanam, S. (2002) The protein kinase complement of the human genome. Science, 298, 1912-1934.
3. Chang, L. and Karin, M. (2001) Mammalian MAP kinase signalling cascades. Nature, 410, 37-40.
4. de Caestecker, M. P., Piek, E. and Roberts, A. B. (2000) Role of transforming growth factor-beta signaling in cancer. J Natl Cancer Inst, 92, 1388-1402.
5. Zhang, H., Berezov, A., Wang, Q., Zhang, G., Drebin, J., Murali, R. and Greene, M. I. (2007) ErbB receptors: from oncogenes to targeted cancer therapies. J Clin Invest, 117, 2051-2058.
6. Rena, G., Pearson, E. R. and Sakamoto, K. (2013) Molecular mechanism of action of metformin: old or new insights? Diabetologia, 56, 1898-1906.
7. Peterson, L. B., Yaffe, M. B. and Imperiali, B. (2014) Selective mitogen activated protein kinase activity sensors through the application of directionally programmable D domain motifs. Biochemistry, 53, 5771-5778.
8. Ni, Q., Titov, D. V. and Zhang, J. (2006) Analyzing protein kinase dynamics in living cells with FRET reporters. Methods, 40, 279-286.
9. Durandau, E., Aymoz, D. and Pelet, S. (2015) Dynamic single cell measurements of kinase activity by synthetic kinase activity relocation sensors. BMC Biol, 13, 55.
10. Lin, W., Mehta, S. and Zhang, J. (2019) Genetically encoded fluorescent biosensors illuminate kinase signaling in cancer. J Biol Chem, 294, 14814-14822.
11. Ulrich, S. M., Kenski, D. M. and Shokat, K. M. (2003) Engineering a “methionine clamp” into Src family kinases enhances specificity toward unnatural ATP analogues. Biochemistry, 42, 7915-7921.
12. Kumar, V., Weng, Y.-C., Geldenhuys, W. J., Wang, D., Han, X., Messing, R. O. and Chou, W.-H. (2015) Generation and characterization of ATP analog-specific protein kinase Cδ. Journal of Biological Chemistry, 290, 1936-1951.
13. Zeliadt, N. (2012) Profile of Kevan M. Shokat. Proceedings of the National Academy of Sciences, 109, 11057-11059.
14. Ultanir, S. K., Yadav, S., Hertz, N. T., Oses-Prieto, J. A., Claxton, S., Burlingame, A. L., Shokat, K. M., Jan, L. Y. and Jan, Y. N. (2014) MST3 kinase phosphorylates TAO1/2 to enable Myosin Va function in promoting spine synapse development. Neuron, 84, 968-982.
15. Allen, J. J., Lazerwith, S. E. and Shokat, K. M. (2005) Bio-orthogonal affinity purification of direct kinase substrates. Journal of the American Chemical Society, 127, 5288-5289.
16. Hindley, A. D., Park, S., Wang, L., Shah, K., Wang, Y., Hu, X., Shokat, K. M., Kolch, W., Sedivy, J. M. and Yeung, K. C. (2004) Engineering the serine/threonine protein kinase Raf-1 to utilise an orthogonal analogue of ATP substituted at the N 6 position. FEBS letters, 556, 26-34.
17. Shah, K. and Shokat, K. M. (2002) A chemical genetic screen for direct v-Src substrates reveals ordered assembly of a retrograde signaling pathway. Chem Biol, 9, 35-47.
18. Hertz, N. T., Wang, B. T., Allen, J. J., Zhang, C., Dar, A. C., Burlingame, A. L. and Shokat, K. M. (2010) Chemical genetic approach for kinase-substrate mapping by covalent capture of thiophosphopeptides and analysis by mass spectrometry. Curr Protoc Chem Biol, 2, 15-36.
19. Dephoure, N., Howson, R. W., Blethrow, J. D., Shokat, K. M. and O'Shea, E. K. (2005) Combining chemical genetics and proteomics to identify protein kinase substrates. Proceedings of the National Academy of Sciences, 102, 17940-17945.
20. Zhang, Y., Kim, W. Y. and Huang, L. (2013) Systemic delivery of gemcitabine triphosphate via LCP nanoparticles for NSCLC and pancreatic cancer therapy. Biomaterials, 34, 3447-3458.
21. Zhang, Y., Peng, L., Mumper, R. J. and Huang, L. (2013) Combinational delivery of c-myc siRNA and nucleoside analogs in a single, synthetic nanocarrier for targeted cancer therapy. Biomaterials, 34, 8459-8468.
22. Manning, B. D. and Cantley, L. C. (2007) AKT/PKB signaling: navigating downstream. Cell, 129, 1261-1274.
23. Bader, A. G., Kang, S., Zhao, L. and Vogt, P. K. (2005) Oncogenic PI3K deregulates transcription and translation. Nat Rev Cancer, 5, 921-929.
24. Engelman, J. A., Luo, J. and Cantley, L. C. (2006) The evolution of phosphatidylinositol 3-kinases as regulators of growth and metabolism. Nat Rev Genet, 7, 606-619.
25. Haas-Kogan, D., Shalev, N., Wong, M., Mills, G., Yount, G. and Stokoe, D. (1998) Protein kinase B (PKB/Akt) activity is elevated in glioblastoma cells due to mutation of the tumor suppressor PTEN/MMAC. Curr Biol, 8, 1195-1198.
26. Maehama, T. and Dixon, J. E. (1998) The tumor suppressor, PTEN/MMAC1, dephosphorylates the lipid second messenger, phosphatidylinositol 3,4,5-trisphosphate. J Biol Chem, 273, 13375-13378.
27. Vivanco, I. and Sawyers, C. L. (2002) The phosphatidylinositol 3-Kinase AKT pathway in human cancer. Nat Rev Cancer, 2, 489-501.
28. Mahajan, K., Coppola, D., Challa, S., Fang, B., Chen, Y. A., Zhu, W., Lopez, A. S., Koomen, J., Engelman, R. W., Rivera, C. et al. (2010) Ack1 mediated AKT/PKB tyrosine 176 phosphorylation regulates its activation. PLoS One, 5, e9646.
29. Testa, J. R. and Tsichlis, P. N. (2005) AKT signaling in normal and malignant cells. Oncogene, 24, 7391-7393.
30. Dan, H. C., Cooper, M. J., Cogswell, P. C., Duncan, J. A., Ting, J. P.-Y. and Baldwin, A. S. (2008) Akt-dependent regulation of NF-κB is controlled by mTOR and Raptor in association with IKK. Genes & development, 22, 1490-1500.
31. Altomare, D. A. and Testa, J. R. (2005) Perturbations of the AKT signaling pathway in human cancer. Oncogene, 24, 7455-7464.
32. Bellacosa, A., Kumar, C. C., Di Cristofano, A. and Testa, J. R. (2005) Activation of AKT kinases in cancer: implications for therapeutic targeting. Adv Cancer Res, 94, 29-86.
33. Danielsen, S. A., Eide, P. W., Nesbakken, A., Guren, T., Leithe, E. and Lothe, R. A. (2015) Portrait of the PI3K/AKT pathway in colorectal cancer. Biochim Biophys Acta, 1855, 104-121.
34. Ericson, K., Gan, C., Cheong, I., Rago, C., Samuels, Y., Velculescu, V. E., Kinzler, K. W., Huso, D. L., Vogelstein, B. and Papadopoulos, N. (2010) Genetic inactivation of AKT1, AKT2, and PDPK1 in human colorectal cancer cells clarifies their roles in tumor growth regulation. Proc Natl Acad Sci USA, 107, 2598-2603.
35. Kirkegaard, T., Witton, C. J., Edwards, J., Nielsen, K. V., Jensen, L. B., Campbell, F. M., Cooke, T. G. and Bartlett, J. M. (2010) Molecular alterations in AKT1, AKT2 and AKT3 detected in breast and prostatic cancer by FISH. Histopathology, 56, 203-211.
36. Mackenzie, R. W. and Elliott, B. T. (2014) Akt/PKB activation and insulin signaling: a novel insulin signaling pathway in the treatment of type 2 diabetes. Diabetes Metab Syndr Obes, 7, 55-64.
37. Okuzumi, T., Ducker, G. S., Zhang, C., Aizenstein, B., Hoffman, R. and Shokat, K. M. (2010) Synthesis and evaluation of indazole based analog sensitive Akt inhibitors. Mol Biosyst, 6, 1389-1402.
38. Okuzumi, T., Fiedler, D., Zhang, C., Gray, D. C., Aizenstein, B., Hoffman, R. and Shokat, K. M. (2009) Inhibitor hijacking of Akt activation. Nat Chem Biol, 5, 484-493.
39. Allen, J. J., Li, M., Brinkworth, C. S., Paulson, J. L., Wang, D., Hubner, A., Chou, W. H., Davis, R. J., Burlingame, A. L., Messing, R. O. et al. (2007) A semisynthetic epitope for kinase substrates. Nat Methods, 4, 511-516.
40. Li, X., Lu, Y., Jin, W., Liang, K., Mills, G. B. and Fan, Z. (2006) Autophosphorylation of Akt at Threonine 72 and Serine 246 A potential mechanism of regulation of Akt kinase activity. Journal of Biological Chemistry, 281, 13837-13843.
41. Li, Y., Wu, Y., Huang, L., Miao, L., Zhou, J., Satterlee, A. B. and Yao, J. (2016) Sigma receptor-mediated targeted delivery of anti-angiogenic multifunctional nanodrugs for combination tumor therapy. Journal of Controlled Release, 228, 107-119.
42. Brattain, M. G., Marks, M. E., McCombs, J., Finely, W. and Brattain, D. E. (1983) Characterization of human colon carcinoma cell lines isolated from a single primary tumour. Br J Cancer, 47, 373-381.
43. Howell, G. M., Humphrey, L. E., Awwad, R. A., Wang, D., Koterba, A., Periyasamy, B., Yang, J., Li, W., Willson, J. K., Ziober, B. L. et al. (1998) Aberrant regulation of transforming growth factor-alpha during the establishment of growth arrest and quiescence of growth factor independent cells. J Biol Chem, 273, 9214-9223.
44. Allen, J. J., Li, M., Brinkworth, C. S., Paulson, J. L., Wang, D., Hübner, A., Chou, W.-H., Davis, R. J., Burlingame, A. L. and Messing, R. O. (2007) A semisynthetic epitope for kinase substrates. Nature methods, 4, 511.
45. Hornbeck, P. V., Zhang, B., Murray, B., Kornhauser, J. M., Latham, V. and Skrzypek, E. (2015) PhosphoSitePlus, 2014: mutations, PTMs and recalibrations. Nucleic Acids Res, 43, D512-520.
46. Guo, Z., Jiang, W., Lages, N., Borcherds, W. and Wang, D. (2014) Relationship between gene duplicability and diversifiability in the topology of biochemical networks. BMC Genomics, 15, 577.
47. Padawer, T., Leighty, R. E. and Wang, D. (2012) Duplicate gene enrichment and expression pattern diversification in multicellularity. Nucleic Acids Res, 40, 7597-7605.
48. Wang, D., Wang, T., Gill, A., Hilliard, T., Chen, F., Karamyshev, A. L. and Zhang, F. (2020) Uncovering the cellular capacity for intensive and specific feedback self-control of the argonautes and MicroRNA targeting activity. Nucleic Acids Res, 48, 4681-4697.
49. Zhang, F. and Wang, D. (2017) The Pattern of microRNA Binding Site Distribution. Genes (Basel), 8, 296.
50. Guo, X. N., Rajput, A., Rose, R., Hauser, J., Beko, A., Kuropatwinski, K., LeVea, C., Hoffman, R. M., Brattain, M. G. and Wang, J. (2007) Mutant PIK3CA-bearing colon cancer cells display increased metastasis in an orthotopic model. Cancer Res, 67, 5851-5858.
51. Kim, A., Lee, J. E., Lee, S. S., Kim, C., Lee, S. J., Jang, W. S. and Park, S. (2013) Coexistent mutations of KRAS and PIK3CA affect the efficacy of NVP-BEZ235, a dual PI3K/MTOR inhibitor, in regulating the PI3K/MTOR pathway in colorectal cancer. Int J Cancer, 133, 984-996.
52. Blethrow, J. D., Glavy, J. S., Morgan, D. O. and Shokat, K. M. (2008) Covalent capture of kinase-specific phosphopeptides reveals Cdk1-cyclin B substrates. Proceedings of the National Academy of Sciences, 105, 1442-1447.
53. Floor, S. N., Barkovich, K. J., Condon, K. J., Shokat, K. M. and Doudna, J. A. (2016) Analog sensitive chemical inhibition of the DEAD-box protein DDX3. Protein Sci, 25, 638-649.
54. Ferguson, K. M. (2008) Structure-based view of epidermal growth factor receptor regulation. Annu Rev Biophys, 37, 353-373.
55. Liu, Q., Zhu, H., Tiruthani, K., Shen, L., Chen, F., Gao, K., Zhang, X., Hou, L., Wang, D., Liu, R. et al. (2018) Nanoparticle-Mediated Trapping of Wnt Family Member 5A in Tumor Microenvironments Enhances Immunotherapy for B-Raf Proto-Oncogene Mutant Melanoma. ACS Nano, 12, 1250-1261.
56. Yang, Y., Li, J., Liu, F. and Huang, L. (2012) Systemic delivery of siRNA via LCP nanoparticle efficiently inhibits lung metastasis. Mol Ther, 20, 609-615.
57. Wu, C., Xu, J., Hao, Y., Zhao, Y., Qiu, Y., Jiang, J., Yu, T., Ji, P. and Liu, Y. (2017) Application of a lipid-coated hollow calcium phosphate nanoparticle in synergistic co-delivery of doxorubicin and paclitaxel for the treatment of human lung cancer A549 cells. Int J Nanomedicine, 12, 7979-7992.

What is claimed is:

1. A method for detecting kinase activity in vivo, comprising:
providing a cell that comprises one or more mutated kinases, wherein the one or more mutated kinases comprise a mutation that enlarges an ATP binding pocket of the kinase;
contacting the cell with an ATP analog-nanoparticle conjugate capable of intracellular delivery of the ATP analog-nanoparticle conjugate, wherein the ATP analog comprises a detectable label; and
culturing the cells under conditions in which the ATP analog-nanoparticle conjugate contacts the one or more mutated kinases in cellulo, wherein the detectable label is transferred from the ATP analog-nanoparticle conjugate to a substrate of the one or more mutated kinases, wherein the one or more kinases react to transfer the detectable label to the substrate.

2. The method of claim 1, wherein at least one of:
the cell is a normal cell, a cell clone, a cell line, a transformed cell, or a transfected cell;
the ATP binding pocket is enlarged by mutating a gate-keeper Methionine residue into a Glycine;
the nanoparticle comprises a liposome, a lipid/calcium/phosphate (LCP) nanoparticle, a polymeric nanoparticle, or a large viral particle;
the one or more mutated kinases is transfected into the cell.
the substrate is transfected into the cell;
the ATP analog is selected from N⁶-phenylethyl-ATP, N⁶-methyl-ATP, N⁶-benzyl-ADP, N⁶-benzyl-ATP, N⁶-phenylpropyl-ATP, or N⁶-phenylbutyl-ATP;
the kinase is an adenylate kinase, a tyrosine kinase, a phosphoinositol kinase, a serine/threonine kinase, a single domain kinase, a double domain kinase, a receptor kinase, a histidine kinase, a dual-specificity kinase, a thermostable kinase, or a cytoplasmic kinase; or
the detectable label is selected from a fluorescence, chemiluminescence, fluorescent energy transfer, radioactive, an enzyme substrate, detection of thiophosphorylation, antigen, or tag.

3. The method of claim 1, wherein the method determines the presence of the substrate in a sample, wherein the substrate is a known substrate for the one or more kinases, or detecting the presence of a previously unknown substrate for the one or more kinases.

4. The method of claim 1, wherein the one or more mutated kinases are selected from at least one of: AGC kinases (PKA, PKG, PKC, PKN, PDK1, AKT, SGK, RSK, RSKR, RSKL, GRK, NDR, MAST, DMPK, YANK, and PTF subfamilies), calcium/calmodulin-dependent protein kinases, casein kinase 1, CMGC kinases (CDK, MAPK, GSK3 and CLK subfamilies), NIMA-related kinase (NEK) kinases, receptor guanylate cyclases (RGC), sterile (STE) kinases, tyrosine protein kinase-like (TKL), tyrosine protein kinase (Tyr), aarF-domain containing kinases (ADCK) kinases, Alpha-type kinases, Fas-activated serine/threonine kinase (FAST) kinases, Pyruvate dehydrogenase kinase PDK/BCKDK kinases, PI3/PI4-kinases, or right open reading frame kinases (RIO) kinases.

5. The method of claim 1, wherein the method further comprises: (i) exposing the kinase coupled to a binding agent specific for the analyte in cellulo, so that a complex is formed between the in vivo kinase and the substrate when present in the cell; (ii) separating complexed in vivo kinase from uncomplexed kinase; wherein the complexed in vivo kinase is contacted simultaneously with ATP and a bioluminescent reagent in cellulo, and (iii) detecting light output from the assay mixture, thereby determining the presence of the analyte in the sample is in cellulo or in vitro.

6. The method of claim 1, further comprising contacting the cell with a kinase inhibitor or a compound suspected of kinase inhibition, and measuring the activity of the in vivo kinase with or without the kinase inhibitor or a compound suspected of kinase inhibition to determine the extent of kinase inhibition.

7. An assay for detecting kinase activity in vivo, comprising:
providing a cell in a well that comprises one or more mutated kinases, wherein the one or more mutated kinases comprise a mutation that enlarges an ATP binding pocket of the kinase;
contacting the cell with an ATP analog-nanoparticle conjugate capable of intracellular delivery of the ATP analog-nanoparticle conjugate, wherein the ATP analog comprises a detectable label;
culturing the cells under conditions in which the ATP analog-nanoparticle conjugate contacts the one or more mutated kinases in cellulo, wherein the detectable label is transferred from the ATP analog-nanoparticle conjugate to a substrate of the one or more mutated kinases; and
detecting the detectable label on the substrate.

8. The assay of claim 7, wherein at least one of:
the cell is a normal cell, a cell clone, a cell line, a transformed cell, or a transfected cell;
the ATP binding pocket is enlarged mutating a gate-keeper Methionine residue into a Glycine;
the nanoparticle comprises a liposome, a lipid/calcium/phosphate (LCP) nanoparticle, a polymeric nanoparticle, or a large viral particle;
the one or more mutated kinases is transfected into the cell.
the substrate is transfected into the cell;
the ATP analog is selected from N⁶-phenylethyl-ATP, N⁶-methyl-ATP, N⁶-benzyl-ADP, N⁶-benzyl-ATP, N⁶-phenylpropyl-ATP, or N⁶-phenylbutyl-ATP;
the kinase is an adenylate kinase, a tyrosine kinase, a phosphoinositol kinase, a serine/threonine kinase, a single domain kinase, a double domain kinase, a receptor kinase, a histidine kinase, a dual-specificity kinase, a thermostable kinase, or a cytoplasmic kinase; or
the detectable label is selected from a fluorescence, chemiluminescence, fluorescent energy transfer, radioactive, an enzyme substrate, detection of thiophosphorylation, antigen, or tag.

9. The assay of claim 7, wherein the method determines the presence of the substrate in a sample, wherein the substrate is a known substrate for the one or more kinases, or detecting the presence of a previously unknown substrate for the one or more kinases.

10. The assay of claim 7, wherein the one or more mutated kinases are selected from at least one of: AGC kinases (PKA, PKG, PKC, PKN, PDK1, AKT, SGK, RSK, RSKR, RSKL, GRK, NDR, MAST, DMPK, YANK, and PTF subfamilies), calcium/calmodulin-dependent protein kinases, casein kinase 1, CMGC kinases (CDK, MAPK, GSK3 and CLK subfamilies), NIMA-related kinase (NEK) kinases, receptor guanylate cyclases (RGC), sterile (STE) kinases, tyrosine protein kinase-like (TKL), tyrosine protein kinase (Tyr), aarF-domain containing kinases (ADCK) kinases, Alpha-type kinases, Fas-activated serine/threonine kinase (FAST) kinases, Pyruvate dehydrogenase kinase PDK/BCKDK kinases, PI3/PI4-kinases, or right open reading frame kinases (RIO) kinases.

11. The assay of claim 7, wherein the method further comprises: (i) exposing the kinase coupled to a binding agent specific for the analyte in cellulo, so that a complex is formed between the in vivo kinase and the substrate when present in the cell; (ii) separating complexed in vivo kinase from uncomplexed kinase; wherein the complexed in vivo kinase is contacted simultaneously with ATP and a bioluminescent reagent in cellulo, and (iii) detecting light output from the assay mixture, thereby determining the presence of the analyte in the sample is in cellulo or in vitro.

12. The assay of claim 7, wherein the assay further comprises contacting the cell with a kinase inhibitor or a compound suspected of kinase inhibition, and measuring the activity of the in vivo kinase with or without the kinase inhibitor or a compound suspected of kinase inhibition to determine the extent of kinase inhibition.

13. A system for detecting kinase activity in vivo, comprising:
a well that comprises cells that comprise one or more mutated kinases, wherein the one or more mutated kinases comprise a mutation that enlarges an ATP binding pocket of the kinase,
wherein the cell is contacted with an ATP analog-nanoparticle conjugate capable of intracellular delivery of the ATP analog-nanoparticle conjugate,
wherein the ATP analog comprises a detectable label, and after a predetermined period of time, detecting a detectable label on a substrate from the ATP analog-nanoparticle conjugate contacts the one or more mutated kinases in cellulo, and
wherein the detectable label was transferred from the ATP analog-nanoparticle conjugate to the substrate of the one or more mutated kinases.

14. The system of claim 13, wherein at least one of:
the cell is a normal cell, a cell clone, a cell line, a transformed cell, or a transfected cell;
the ATP binding pocket is enlarged by mutating a gate-keeper Methionine residue into a Glycine;
the nanoparticle comprises a liposome, a lipid/calcium/phosphate (LCP) nanoparticle, a polymeric nanoparticle, or a large viral particle;
the one or more mutated kinases is transfected into the cell.
the substrate is transfected into the cell;
the ATP analog is selected from N⁶-phenylethyl-ATP, N⁶-methyl-ATP, N⁶-benzyl-ADP, N⁶-benzyl-ATP, N⁶-phenylpropyl-ATP, or N⁶-phenylbutyl-ATP;
the kinase is an adenylate kinase, a tyrosine kinase, a phosphoinositol kinase, a serine/threonine kinase, a single domain kinase, a double domain kinase, a receptor kinase, a histidine kinase, a dual-specificity kinase, a thermostable kinase, or a cytoplasmic kinase; or
the detectable label is selected from a fluorescence, chemiluminescence, fluorescent energy transfer, radioactive, an enzyme substrate, detection of thiophosphorylation, antigen, or tag.

15. The system of claim 13, wherein the system determines the presence of the substrate in a sample, wherein the substrate is a known substrate for the one or more kinases, or detecting the presence of a previously unknown substrate for the one or more kinases.

16. The system of claim 13, wherein the one or more mutated kinases are selected from at least one of: AGC kinases (PKA, PKG, PKC, PKN, PDK1, AKT, SGK, RSK, RSKR, RSKL, GRK, NDR, MAST, DMPK, YANK, and PTF subfamilies), calcium/calmodulin-dependent protein kinases, casein kinase 1, CMGC kinases (CDK, MAPK, GSK3 and CLK subfamilies), NIMA-related kinase (NEK) kinases, receptor guanylate cyclases (RGC), sterile (STE) kinases, tyrosine protein kinase-like (TKL), tyrosine protein kinase (Tyr), aarF-domain containing kinases (ADCK) kinases, Alpha-type kinases, Fas-activated serine/threonine kinase (FAST) kinases, Pyruvate dehydrogenase kinase PDK/BCKDK kinases, PI3/PI4-kinases, or right open reading frame kinases (RIO) kinases.

17. The system of claim 13, wherein the system: (i) exposes the kinase coupled to a binding agent specific for the analyte in cellulo, so that a complex is formed between the in vivo kinase and the substrate when present in the cell; (ii) separates complexed in vivo kinase from uncomplexed kinase; wherein the complexed in vivo kinase is contacted simultaneously with ATP and a bioluminescent reagent in cellulo, and (iii) detects light output from the assay mixture, thereby determining the presence of the analyte in the sample is in cellulo or in vitro.

18. The system of claim 13, further comprising contacting the cell with a kinase inhibitor or a compound suspected of kinase inhibition, and measuring the activity of the in vivo kinase with or without the kinase inhibitor or a compound suspected of kinase inhibition to determine the extent of kinase inhibition.

19. The system of claim 13, wherein the system comprises at least one of:
an array comprising two or more wells, wherein each of the wells comprises a different mutated kinase, wherein each well is used to detect an activity of a different mutated kinase on the one or more substrate;
an array comprising two or more wells, wherein each of the wells comprises a different substrate, wherein each well is used to detect an activity of a different substrate by the one or more mutated kinases, or both.

20. A kit for detecting kinase activity in vivo, comprising:
a well that comprises cells that comprise one or more mutated kinases, wherein the one or more mutated kinases comprise a mutation that enlarges an ATP binding pocket of the kinase; and
an ATP analog-nanoparticle conjugate capable of intracellular delivery of the ATP analog-nanoparticle conjugate, wherein the cell is contacted with the ATP analog-nanoparticle conjugate, and wherein the ATP analog-nanoparticle comprises a detectable label, and after a predetermined period of time, detecting a detectable label on a substrate from the ATP analog-nanoparticle conjugate contacts the one or more mutated kinases in cellulo, and wherein the detectable label was transferred from the ATP analog-nanoparticle conjugate to the substrate of the one or more mutated kinases.
